(19) United States
(12) Patent Application Publication
(10) Pub. No.: US 2022/0280517 A1
(43) Pub. Date: Sep. 8, 2022

(54) ORGANIC COMPOUNDS

(71) Applicant: INTRA-CELLULAR THERAPIES, INC., New York, NY (US)

(72) Inventors: Robert E. DAVIS, San Diego, CA (US); Peng LI, New Milford, NJ (US)

(73) Assignee: INTRA-CELLULAR THERAPIES, INC., New York, NY (US)

(21) Appl. No.: 17/636,645

(22) PCT Filed: Aug. 21, 2020
(86) PCT No.: PCT/US2020/047451

§ 371 Date: Feb. 18, 2022

Related U.S. Application Data
(60) Provisional application No. 62/890,162, filed on Aug. 22, 2019.

(51) Int. Cl. A61K 31/519; A61P 13/12

(52) U.S. Cl. CPC A61K 31/519; A61P 13/12

(57) ABSTRACT

The disclosure relates to methods of administering comprising administration of inhibitors of phosphodiesterase 1 (PDE1) for the treatment and/or prophylaxis of renal disorders, such as chronic kidney disease. Related compounds and methods of making are further defined.

### FIELD OF DISCLOSURE

The field relates to inhibitors of phosphodiesterase 1 (PDE1) useful for the treatment of renal disorders, such as chronic kidney disease. The field further relates to the administration of inhibitors of phosphodiesterase 1 (PDE1) for the for the treatment of renal disorders, such as chronic kidney disease, or for the treatment of related conditions characterized by an increased expression in PDE1.

### BACKGROUND OF THE DISCLOSURE

Kidney fibrosis is an important factor for the progression of kidney diseases, such as diabetes mellitus induced kidney failure, glomerulosclerosis and nephritis resulting in chronic kidney disease or end-stage renal disease. Cyclic adenosine monophosphate (cAMP) and cyclic guanosine monophosphate (cGMP) have been implicated to suppress several known renal diseases through a number of complex mechanisms, such as the nitric oxide/ANP/guanylyl cyclases/cGMP-dependent protein kinase and cAMP/Epac/adenylyl cyclases/cAMP-dependent protein kinase pathways. From these diverse mechanisms it has been proposed that new pharmacological treatments will evolve for the therapy or even prevention of kidney failure.

Renal fibrosis is commonly found in chronic kidney diseases (CKD), e.g., diabetic nephropathy, glomerulosclerosis and lupus nephritis. Such CKDs can be caused by oxidative stress, hypoxia, inflammation, autoimmune disease or altered metabolism. Acute insult of the kidney by ischemia or toxins can also finally result in CKD. However, there are common disease patterns in various CKDs, as the formation of myofibroblasts (which secrete in turn extracellular matrix (ECM) proteins) is regularly the first step in the progression of fibrosis.

Interference of profibrotic signalling pathways is believed to be a useful tool for disease suppression. For example, inhibitors of TGFβ or of its signalling pathways, preventing the myofibroblast differentiation, are valuable as antifibrotic agents. Expression of TGFβ can be reduced e.g., by pirfenidone, which might be effective for treatment of diabetic kidney disease. Additionally, it improves oxidative stress induced by mitochondrial dysfunction. Signalling of cyclic nucleotides can act on several parts of these fibrotic processes as they suppress, e.g., interstitial fibrosis via reduced TGFβ signalling and myofibroblast formation or reduction of oxidative stress.

Eleven families of phosphodiesterases (PDEs) have been identified but only PDEs in Family I, the Ca2+/calmodulin-dependent phosphodiesterases (CaM-PDEs), which are activated by Ca2+/calmodulin and have been shown to mediate the calcium and cyclic nucleotide (e.g. cGMP and cAMP) signaling pathways. The three known CaM-PDE genes, PDE1A, PDE1B, and PDE1C, are all expressed in central nervous system tissue. PDE1A is expressed in the brain, lung and heart. PDE1B is primarily expressed in the central nervous system, but it is also detected in monocytes and neutrophils and has been shown to be involved in inflammatory responses of these cells. PDE1C is expressed in olfactory epithelium, cerebellar granule cells, striatum, heart, and vascular smooth muscle. PDE1C has been demonstrated to be a major regulator of smooth muscle proliferation in human smooth muscle.

Cyclic nucleotide phosphodiesterases down-regulate intracellular cAMP and cGMP signaling by hydrolyzing these cyclic nucleotides to their respective 5′-monophosphates (5′AMP and 5′GMP), which are inactive in terms of intra-cellular signaling pathways. Both cAMP and cGMP are central intracellular second-messengers and they play roles in regulating numerous cellular functions. PDE1A and PDE1B preferentially hydrolyze cGMP over cAMP, while PDE1C shows approximately equal cGMP and cAMP hydrolysis.

In cardiac fibroblasts, PDE1A is highly upregulated after stimulation with ATII and TGFβ. Moreover, PDE1 inhibitors have been reported to decrease ATII or TGFβ induced cardiac myofibroblast activation, ECM production, and profibrotic gene expression, suggesting that PDE1 inhibition also mediates the antifibrotic effects via cAMP. The PDE1 isozymes are abundant in the kidney. Thus, it follows that increased cAMP levels induced by specific PDE1 inhibitors could be beneficial in treating renal diseases.

There is a need for additional treatment choices for patients suffering from chronic kidney disease, particularly diabetic kidney disease. New, safer and selective strategies for modulating cAMP in cancer cells are needed.

### SUMMARY OF THE DISCLOSURE

Presented herein are compounds and methods for the treatment of a renal disorder, e.g., kidney fibrosis, chronic kidney disease, kidney fibrosis, renal failure, glomerulosclerosis and nephritis. Studies have shown that cyclic nucleotides cAMP and cGMP play a prominent role in progressing such renal disorders. The compounds of the present disclosure are potent inhibitors of PDE 1.

In various embodiments, the present disclosure provides for methods for the treatment or prophylaxis of a renal disorder, e.g., kidney fibrosis, chronic kidney disease, kidney fibrosis, renal failure, glomerulosclerosis and nephritis comprising administering a pharmaceutically acceptable amount of a PDE1 inhibitor as disclosed herein to a subject in need thereof. In some embodiments, the renal disorder is chronic kidney disease. In some embodiments, the chronic kidney disease is consequent to diabetes, an injury to a kidney, high blood pressure, cancer growth (e.g., polycystic kidney disease), or a cardiovascular disorder (e.g. angina, stroke, essential hypertension, pulmonary hypertension, secondary hypertension, isolated systolic hypertension, hypertension associated with diabetes, hypertension associated with atherosclerosis, renovascular hypertension, congestive heart failure, myocardial, angina, and stroke, hypertension, an inflammatory disease or disorder, fibrosis, cardiac hypertrophy, vascular remodeling, and an connective tissue disease or disorder, e.g., Marfan Syndrome).

### DETAILED DESCRIPTION OF THE DISCLOSURE

### Compounds for Use in the Methods of the Disclosure

In one embodiment, the PDE1 inhibitors for use in the methods of treatment and prophylaxis described herein are selective PDE1 inhibitors.

### PDE1 Inhibitors

In one embodiment the invention provides that the PDE1 inhibitors for use in the methods described herein are compounds of Formula I:

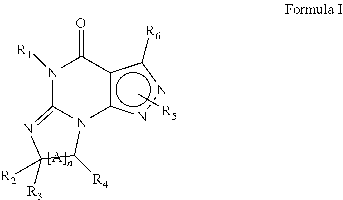

wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero)arylalkoxy, or (optionally hetero)arylalkyl; or R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl; or R₅ is attached to one of the nitrogens on the pyrazolo portion of Formula I and is a moiety of Formula A

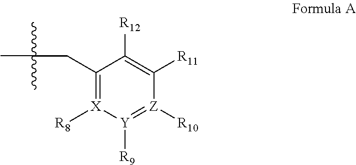

wherein X, Y and Z are, independently, N or C, and R₈, R₉, R₁₁ and R₁₂ are independently H or halogen (e.g., Cl or F), and R₁₀ is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl (e.g., benzyl), arylamino (e.g., phenylamino), heterarylamino, N,N-dialkylamino, N,N-diarylamino, or N-aryl-N-(arylakyl)amino (e.g., N-phenyl-N-(1,1′-biphen-4-ylmethyl)amino); and
(v) n=0 or 1;
(vi) when n=1, A is —C(R₁₃R₁₄)—
wherein R₁₃ and R₁₄, are, independently, H or C₁₋₄ alkyl, aryl, heteroaryl, (optionally hetero)arylalkoxy or (optionally hetero)arylalkyl;
in free, salt or prodrug form, including its enantiomers, diastereoisomers and racemates.

In another embodiment the invention provides that the PDE1 inhibitors for use in the methods as described herein are Formula 1a:

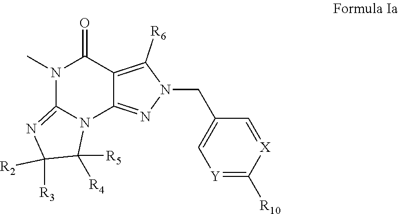

wherein
(i) R₂ and R₅ are independently H or hydroxy and R₃ and R₄ together form a tri- or tetra-methylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref. the carbon carrying R₃ having the R configuration];
(ii) R₆ is (optionally halo- or hydroxy-substituted) phenylamino, (optionally halo- or hydroxy-substituted) benzylamino, C₁₋₄alkyl, or C₁₋₄alkyl sulfide; for example, phenylamino or 4-fluorophenylamino;
(iii) R₁₀ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and X and Y are independently C or N,
in free, pharmaceutically acceptable salt or prodrug form, including its enantiomers, diastereoisomers and racemates.

In another embodiment the invention provides that the PDE1 inhibitors for use in the methods as described herein are compounds of Formula II:

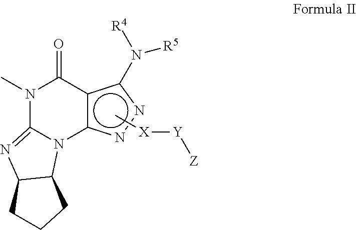

(i) X is C₁₋₆alkylene (e.g., methylene, ethylene or prop-2-yn-1-ylene);
(ii) Y is a single bond, alkynylene (e.g., —C≡C—), arylene (e.g., phenylene) or heteroarylene (e.g., pyridylene);
(iii) Z is H, aryl (e.g., phenyl), heteroaryl (e.g., pyridyl, e.g., pyrid-2-yl), halo (e.g., F, Br, Cl), haloC₁₋₆alkyl (e.g., trifluoromethyl), —C(O)—R¹, —N(R²)(R³), or C₃₋₇cycloalkyl optionally containing at least one atom selected from a group consisting of N or O (e.g., cyclopentyl, cyclohexyl, tetrahydro-2H-pyran-4-yl, or morpholinyl);
(iv) R¹is C₁₋₆alkyl, haloC₁₋₆alkyl, —OH or —OC₁₋₆alkyl (e.g., —OCH₃);
(v) R² and R³ are independently H or C₁₋₆alkyl;
(vi) R⁴ and R⁵ are independently H, C₁₋₆alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆alkoxy;
(vii) wherein X, Y and Z are independently and optionally substituted with one or more halo (e.g., F, Cl or Br), C₁₋₆alkyl (e.g., methyl), haloC₁₋₆alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl),

in free, salt or prodrug form.

In yet another embodiment the invention provides that the PDE1 inhibitors for use in the methods as described herein are Formula III:

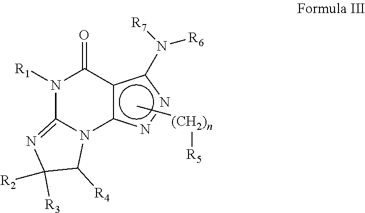

wherein
(i) R₁ is H or C₁₋₄ alkyl (e.g., methyl or ethyl);
(ii) R₂ and R₃ are independently H or C₁₋₆ alkyl (e.g., methyl or ethyl);
(iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆ alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆alkyl (e.g., methyl or ethyl) and halogen (e.g., F or Cl), for example unsubstituted phenyl or phenyl substituted with one or more halogen (e.g., F) or phenyl substituted with one or more C₁₋₆ alkyl and one or more halogen or phenyl substituted with one C₁₋₆ alkyl and one halogen, for example 4-fluorophenyl or 3,4-difluorophenyl or 4-fluoro-3-methylphenyl; and
(vi) n is 1, 2, 3, or 4,

in free or salt form.

In yet another embodiment the invention provides that the PDE1 inhibitors for use in the methods as described herein are Formula IV

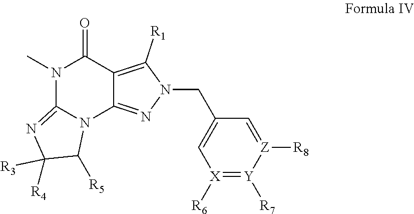

in free or salt form, wherein
(i) R₁ is C₁₋₄alkyl (e.g., methyl or ethyl), or —NH(R₂), wherein R₂ is phenyl optionally substituted with halo (e.g., fluoro), for example, 4-fluorophenyl;
(ii) X, Y and Z are, independently, N or C;
(iii) R₃, R₄ and R₅ are independently H or C₁₋₄alkyl (e.g., methyl); or R₃ is H and R₄ and R₅ together form a tri-methylene bridge (pref. wherein the R₄ and R₅ together have the cis configuration, e.g., where the carbons carrying R₄ and R₅ have the R and S configurations, respectively),
(iv) R₆, R₇ and R₈ are independently:
H,
C₁₋₄alkyl (e.g., methyl),
pyrid-2-yl substituted with hydroxy, or
—S(O)₂—NH₂;
   (v) Provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy.

In another embodiment the invention provides that the PDE1 inhibitors for use in the methods as described herein are Formula V:

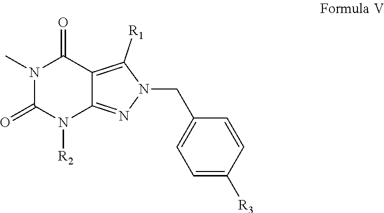

wherein

(i) R₁ is —NH(R₄), wherein R₄ is phenyl optionally substituted with halo (e.g., fluoro), for example, 4-fluorophenyl;

(ii) R₂ is H or C₁₋₆alkyl (e.g., methyl, isobutyl or neopentyl);

(iii) R₃ is —SO₂NH₂ or —COOH;

in free or salt form.

In another embodiment the invention provides that the PDE1 inhibitors for use in the methods as described herein are Formula VI:

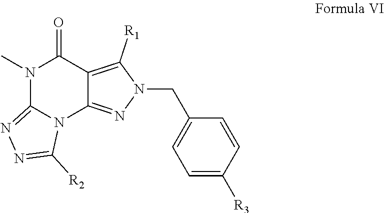

wherein

(i) R₁ is —NH(R₄), wherein R₄ is phenyl optionally substituted with halo (e.g., fluoro), for example, 4-fluorophenyl;

(ii) R₂ is H or C₁₋₆alkyl (e.g., methyl or ethyl);

(iii) R₃ is H, halogen (e.g., bromo), C₁₋₆alkyl (e.g., methyl), aryl optionally substituted with halogen (e.g., 4-fluorophenyl), heteroaryl optionally substituted with halogen (e.g., 6-fluoropyrid-2-yl or pyrid-2-yl), or acyl (e.g., acetyl),

in free or salt form.

In one embodiment, the present disclosure provides for administration of a PDE1 inhibitor for use in the methods described herein (e.g., a compound according to Formulas I, Ia, II, III, IV, V, and/or VI), wherein the inhibitor is a compound according to the following:

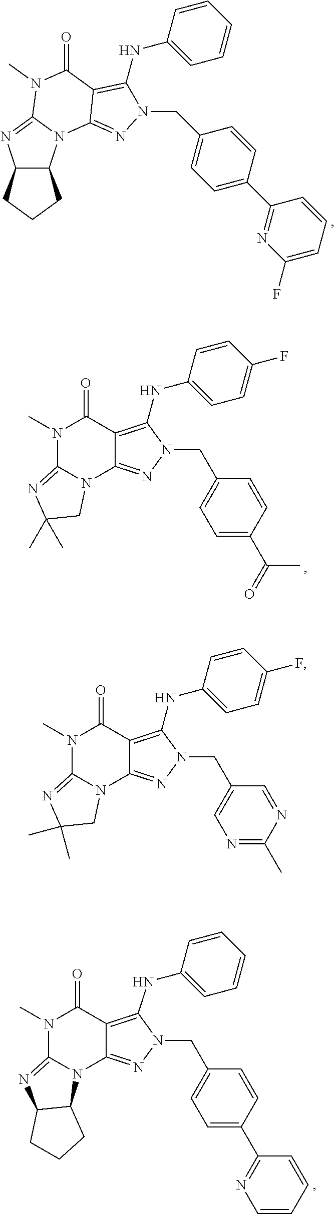

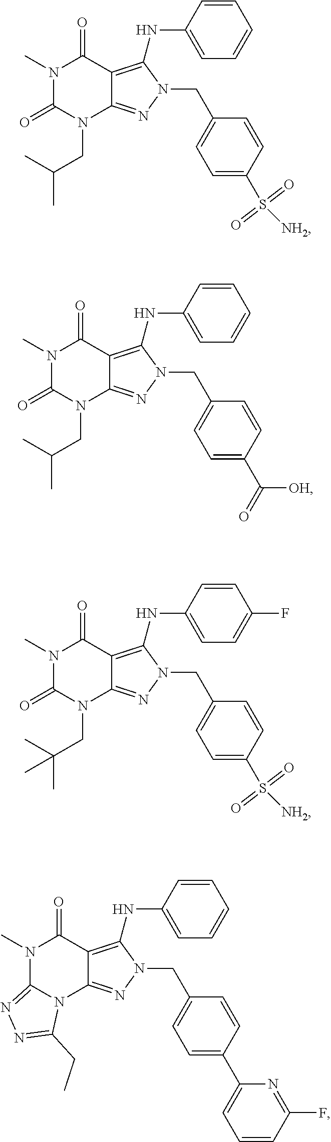 [SMILES: CC(C)Cn1c(=O)n(C)c(=O)c2c(Nc3ccccc3)n(Cc3ccc(S(N)(=O)=O)cc3)nc21 ; CC(C)Cn1c(=O)n(C)c(=O)c2c(Nc3ccccc3)n(Cc3ccc(C(=O)O)cc3)nc21 ; Cn1c(=O)c2c(Nc3ccc(F)cc3)n(Cc3ccc(S(N)(=O)=O)cc3)nc2n(CC(C)(C)C)c1=O ; CCc1nnc2n(C)c(=O)c3c(Nc4ccccc4)n(Cc4ccc(-c5cccc(F)n5)cc4)nc3n12]

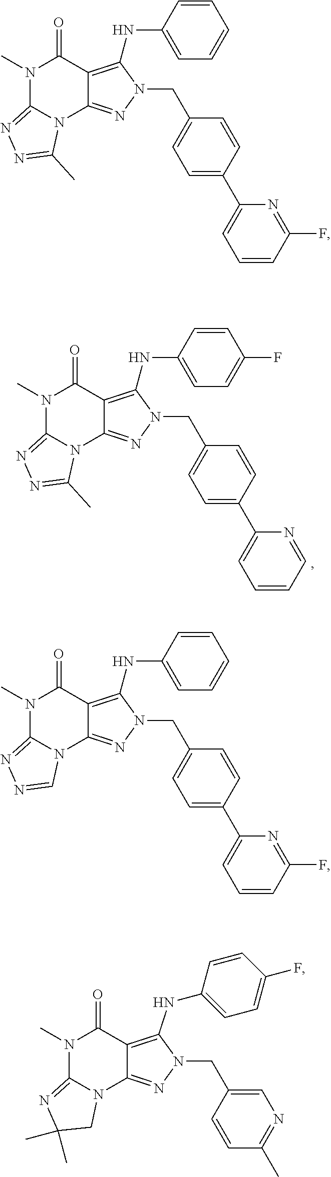 [SMILES: Cc1nnc2n(C)c(=O)c3c(Nc4ccccc4)n(Cc4ccc(-c5cccc(F)n5)cc4)nc3n12 ; Cc1nnc2n(C)c(=O)c3c(Nc4ccc(F)cc4)n(Cc4ccc(-c5ccccn5)cc4)nc3n12 ; Cn1c(=O)c2c(Nc3ccccc3)n(Cc3ccc(-c4cccc(F)n4)cc3)nc2n2cnnc12 ; Cc1ccc(Cn2nc3c(c2Nc2ccc(F)cc2)C(=O)N(C)C2=NC(C)(C)CN23)cn1]

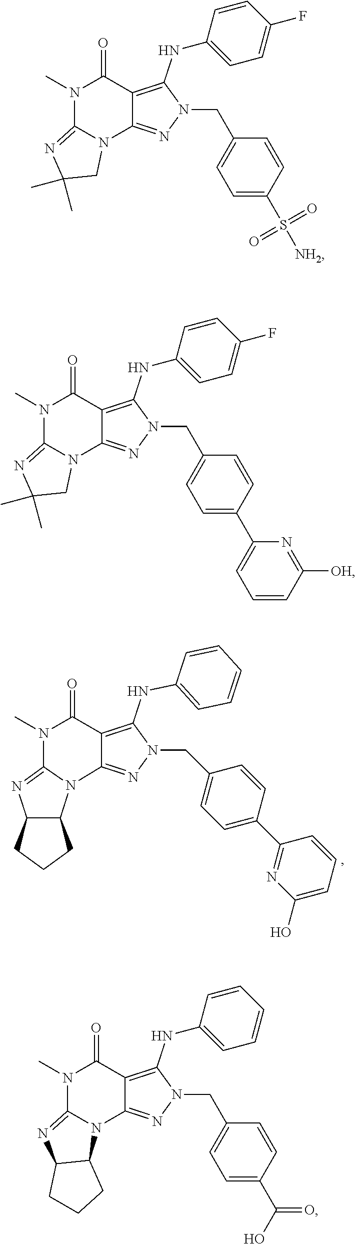 [SMILES: CN1C(=O)c2c(nn(Cc3ccc(S(N)(=O)=O)cc3)c2Nc2ccc(F)cc2)N2CC(C)(C)N=C12 ; CN1C(=O)c2c(nn(Cc3ccc(-c4cccc(O)n4)cc3)c2Nc2ccc(F)cc2)N2CC(C)(C)N=C12 ; CN1C(=O)c2c(nn(Cc3ccc(-c4cccc(O)n4)cc3)c2Nc2ccccc2)N2C1=N[C@@H]1CCC[C@@H]12 ; CN1C(=O)c2c(nn(Cc3ccc(C(=O)O)cc3)c2Nc2ccccc2)N2C1=N[C@@H]1CCC[C@@H]12]

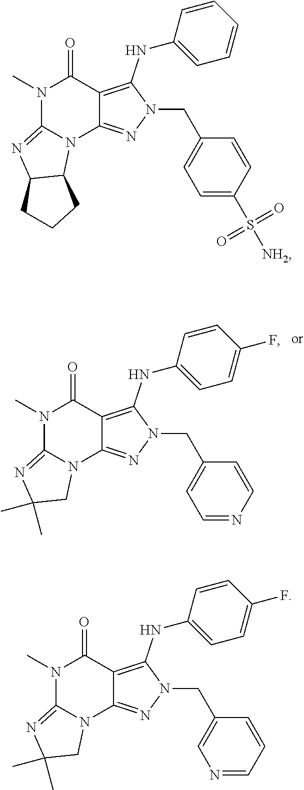 [SMILES: CN1C(=O)c2c(nn(Cc3ccc(S(N)(=O)=O)cc3)c2Nc2ccccc2)N2C1=N[C@@H]1CCC[C@@H]12 ; CN1C(=O)c2c(nn(Cc3ccncc3)c2Nc2ccc(F)cc2)N2CC(C)(C)N=C12 ; "or" ; CN1C(=O)c2c(nn(Cc3cccnc3)c2Nc2ccc(F)cc2)N2CC(C)(C)N=C12]

In one embodiment the invention provides administration of a PDE1 inhibitor for use in the methods as described herein, wherein the inhibitor is a compound according to the following:

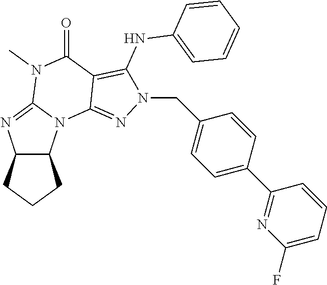 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.

In still another embodiment, the invention provides administration of a PDE1 inhibitor for use in the methods as described herein, wherein the inhibitor is a compound according to the following:

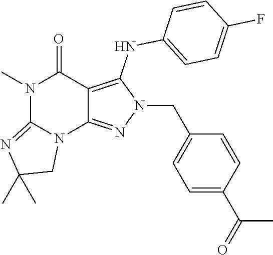 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.

In still another embodiment, the invention provides administration of a PDE1 inhibitor for use in the methods as described herein, wherein the inhibitor is a compound according to the following:

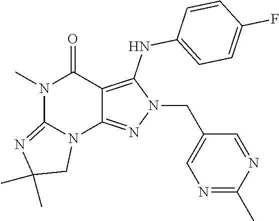 [SMILES: Cc1ncc(Cn2nc3c(c2Nc2ccc(F)cc2)C(=O)N(C)C2=NC(C)(C)CN23)cn1]

in free or pharmaceutically acceptable salt form.

In still another embodiment, the invention provides administration of a PDE1 inhibitor for use in the methods as described herein, wherein the inhibitor is a compound according to the following:

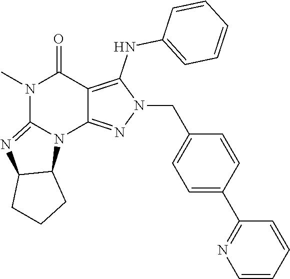 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.

In one embodiment, selective PDE1 inhibitors of the any of the preceding formulae (e.g., Formulas I, Ia, II, III, IV, V, and/or VI) are compounds that inhibit phosphodiesterase-mediated (e.g., PDE1-mediated, especially PDE1B-mediated) hydrolysis of cGMP, e.g., the preferred compounds have an IC₅₀ of less than 1 μM, preferably less than 500 nM, preferably less than 50 nM, and preferably less than 5 nM in an immobilized-metal affinity particle reagent PDE assay, in free or salt form.

In other embodiments, the invention provides administration of a PDE1 inhibitor for treatment of a condition selected from a cancer or tumor; for inhibiting the proliferation, migration and/or invasion of tumorous cells; and/or for treating a glioma, wherein the inhibitor is a compound according to the following:

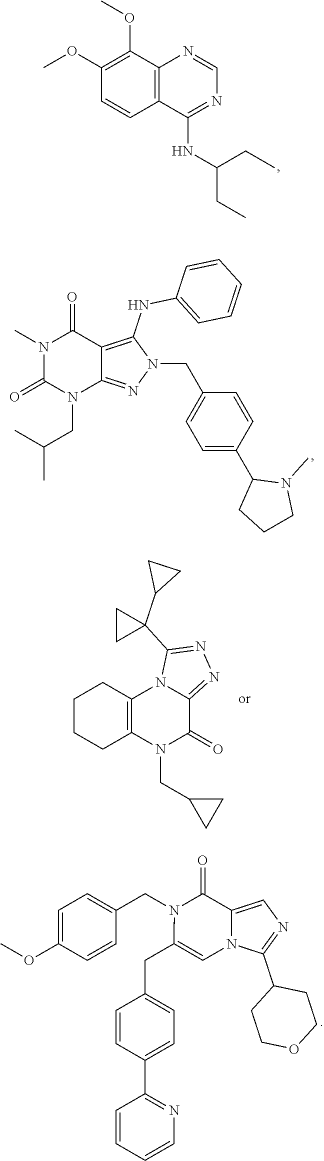

Further examples of PDE1 inhibitors suitable for use in the methods and treatments discussed herein can be found in International Publication WO2006133261A2; U.S. Pat. Nos. 8,273,750; 9,000,001; 9,624,230; International Publication WO2009075784A1; U.S. Pat. Nos. 8,273,751; 8,829,008; 9,403,836; International Publication WO2014151409A1, U.S. Pat. Nos. 9,073,936; 9,598,426; 9,556,186; U.S. Publication 2017/0231994A1, International Publication WO2016022893A1, and U.S. Publication 2017/0226117A1, each of which are incorporated by reference in their entirety.

Still further examples of PDE1 inhibitors suitable for use in the methods and treatments discussed herein can be found in International Publication WO2018007249A1; U.S. Publication 2018/0000786; International Publication WO2015118097A1; U.S. Pat. No. 9,718,832; International Publication WO2015091805A1; U.S. Pat. No. 9,701,665; U.S. Publication 2015/0175584A1; U.S. Publication 2017/0267664A1; International Publication WO2016055618A1; U.S. Publication 2017/0298072A1; International Publication WO2016170064A1; U.S. Publication 2016/0311831A1; International Publication WO2015150254A1; U.S. Publication 2017/0022186A1; International Publication WO2016174188A1; U.S. Publication 2016/0318939A1; U.S. Publication 2017/0291903A1; International Publication WO2018073251A1; International Publication WO2017178350A1; and U.S. Publication 2017/0291901A1; each of which are incorporated by reference in their entirety. In any situation in which the statements of any documents incorporated by reference contradict or are incompatible with any statements made in the present disclosure, the statements of the present disclosure shall be understood as controlling.

If not otherwise specified or clear from context, the following terms herein have the following meanings:
(a) “Selective PDE1 inhibitor” as used herein refers to a PDE1 inhibitor with at least 100-fold selectivity for PDE1 inhibition over inhibition of any other PDE isoform.
(b) “Alkyl” as used herein is a saturated or unsaturated hydrocarbon moiety, preferably saturated, preferably having one to six carbon atoms, which may be linear or branched, and may be optionally mono-, di- or tri- substituted, e.g., with halogen (e.g., chloro or fluoro), hydroxy, or carboxy.
(c) “Cycloalkyl” as used herein is a saturated or unsaturated nonaromatic hydrocarbon moiety, preferably saturated, preferably comprising three to nine carbon atoms, at least some of which form a nonaromatic mono- or bicyclic, or bridged cyclic structure, and which may be optionally substituted, e.g., with halogen (e.g., chloro or fluoro), hydroxy, or carboxy. Wherein the cycloalkyl optionally contains one or more atoms selected from N and O and/or S, said cycloalkyl may also be a heterocycloalkyl.
(d) “Heterocycloalkyl” is, unless otherwise indicated, saturated or unsaturated nonaromatic hydrocarbon moiety, preferably saturated, preferably comprising three to nine carbon atoms, at least some of which form a nonaromatic mono- or bicyclic, or bridged cyclic structure, wherein at least one carbon atom is replaced with N, O or S, which heterocycloalkyl may be optionally substituted, e.g., with halogen (e.g., chloro or fluoro), hydroxy, or carboxy.
(e) “Aryl” as used herein is a mono or bicyclic aromatic hydrocarbon, preferably phenyl, optionally substituted, e.g., with alkyl (e.g., methyl), halogen (e.g., chloro or fluoro), haloalkyl (e.g., trifluoromethyl), hydroxy, carboxy, or an additional aryl or heteroaryl (e.g., biphenyl or pyridylphenyl).
(f) “Heteroaryl” as used herein is an aromatic moiety wherein one or more of the atoms making up the aromatic ring is sulfur or nitrogen rather than carbon, e.g., pyridyl or thiadiazolyl, which may be optionally substituted, e.g., with alkyl, halogen, haloalkyl, hydroxy or carboxy.

Compounds of the Disclosure, e.g., PDE1 inhibitors as described herein, may exist in free or salt form, e.g., as acid addition salts. In this specification unless otherwise indicated, language such as “Compounds of the Disclosure” is to be understood as embracing the compounds in any form, for example free or acid addition salt form, or where the compounds contain acidic substituents, in base addition salt form. The Compounds of the Disclosure are intended for use as pharmaceuticals, therefore pharmaceutically acceptable salts are preferred. Salts which are unsuitable for pharmaceutical uses may be useful, for example, for the isolation or purification of free Compounds of the Disclosure or their pharmaceutically acceptable salts, are therefore also included.

Compounds of the Disclosure may in some cases also exist in prodrug form. A prodrug form is compound which converts in the body to a Compound of the Disclosure. For example, when the Compounds of the Disclosure contain hydroxy or carboxy substituents, these substituents may form physiologically hydrolysable and acceptable esters. As used herein, “physiologically hydrolysable and acceptable ester” means esters of Compounds of the Disclosure which are hydrolysable under physiological conditions to yield acids (in the case of Compounds of the Disclosure which have hydroxy substituents) or alcohols (in the case of Compounds of the Disclosure which have carboxy substituents) which are themselves physiologically tolerable at doses to be administered. Therefore, wherein the Compound of the Disclosure contains a hydroxy group, for example, Compound-OH, the acyl ester prodrug of such compound, i.e., Compound-O—C(O)—C₁₋₄alkyl, can hydrolyze in the body to form physiologically hydrolysable alcohol (Compound-OH) on the one hand and acid on the other (e.g., HOC(O)—C₁₋₄alkyl). Alternatively, wherein the Compound of the Disclosure contains a carboxylic acid, for example, Compound-C(O)OH, the acid ester prodrug of such compound, Compound-C(O)O—C1-4alkyl can hydrolyze to form Compound-C(O)OH and HO—C1-4alkyl. As will be appreciated the term thus embraces conventional pharmaceutical prodrug forms.

In another embodiment, the disclosure further provides a pharmaceutical composition comprising a PDE1 inhibitor in combination with an additional therapeutic agent, each in free or pharmaceutically acceptable salt form, in admixture with a pharmaceutically acceptable carrier. The term “combination,” as used herein, embraces simultaneous, sequential, or contemporaneous administration of the PDE1 inhibitor and the additional therapeutic agent. In another embodiment, the disclosure provides a pharmaceutical composition containing such a compound.

### Methods of Using Compounds of the Disclosure

In another embodiment, the present application provides for a method (Method 1) for the treatment or prophylaxis of a renal disorder comprising administering a pharmaceutically acceptable amount of a PDE1 inhibitor (i.e., PDE1 inhibitor according to Formulas I, Ia, II, III, IV, V, and/or VI) to a subject in need thereof.
1.1 Method 1, wherein the renal disorder is selected from one or more of kidney fibrosis, chronic kidney disease, renal failure, glomerulosclerosis and nephritis.
1.2 Any preceding Method, wherein the condition is kidney fibrosis.
1.3 Any preceding Method, wherein the condition is chronic kidney disease.
1.4 Any preceding Method, wherein the condition is renal failure.
1.5 Any preceding Method, wherein the condition is glomerulosclerosis.
1.6 Any preceding Method, wherein the condition is nephritis.
1.7 Any preceding Method, wherein the renal disorder is consequent to diabetes, an injury to a kidney, high blood pressure, a cancerous growth (e.g., polycystic kidney disease), or a cardiovascular disorder (e.g. angina, stroke, essential hypertension, pulmonary hypertension, secondary hypertension, isolated systolic hypertension, hypertension associated with diabetes, hypertension associated with atherosclerosis, renovascular hypertension, congestive heart failure, myocardial, angina, and stroke, hypertension, an inflammatory disease or disorder, fibrosis, cardiac hypertrophy, vascular remodeling, and an connective tissue disease or disorder, e.g., Marfan Syndrome).
1.8 Any preceding Method, wherein the renal disorder is consequent to diabetes.
1.9 Any of Methods 1-1.7, wherein the renal disorder is consequent to an injury to a kidney.
1.10 Any of Methods 1-1.7, wherein the renal disorder is consequent to high blood pressure.
1.11 Any of Methods 1-1.7, wherein the renal disorder is consequent to a cancerous growth (e.g., polycystic kidney disease).
1.12 Any of Methods 1-1.7, wherein the renal disorder is consequent to a cardiovascular disorder (e.g. angina, stroke, essential hypertension, pulmonary hypertension, secondary hypertension, isolated systolic hypertension, hypertension associated with diabetes, hypertension associated with atherosclerosis, renovascular hypertension, congestive heart failure, myocardial, angina, and stroke, hypertension, an inflammatory disease or disorder, fibrosis, cardiac hypertrophy, vascular remodeling, and an connective tissue disease or disorder, e.g., Marfan Syndrome).
1.13 Methods 1 or 1.1, wherein the renal disorder is chronic kidney disease consequent to diabetes.
1.14 Methods 1 or 1.1, wherein the renal disorder is chronic kidney disease consequent to an injury to a kidney.
1.15 Methods 1 or 1.1, wherein the renal disorder is chronic kidney disease consequent to high blood pressure.
1.16 Methods 1 or 1.1, wherein the renal disorder is chronic kidney disease consequent to a cancerous growth (e.g., polycystic kidney disease).
1.17 Methods 1 or 1.1, wherein the renal disorder is chronic kidney disease consequent to a cardiovascular disorder (e.g. angina, stroke, essential hypertension, pulmonary hypertension, secondary hypertension, isolated systolic hypertension, hypertension associated with diabetes, hypertension associated with atherosclerosis, renovascular hypertension, congestive heart failure, myocardial, angina, and stroke, hypertension, an inflammatory disease or disorder, fibrosis, cardiac hypertrophy, vascular remodeling, and an connective tissue disease or disorder, e.g., Marfan Syndrome).
1.18 Any of the preceding Methods, wherein the PDE1 inhibitor is a PDE1 inhibitor according to Formulas I, Ia, II, III, IV, V, and/or VI or a compound according to the following:

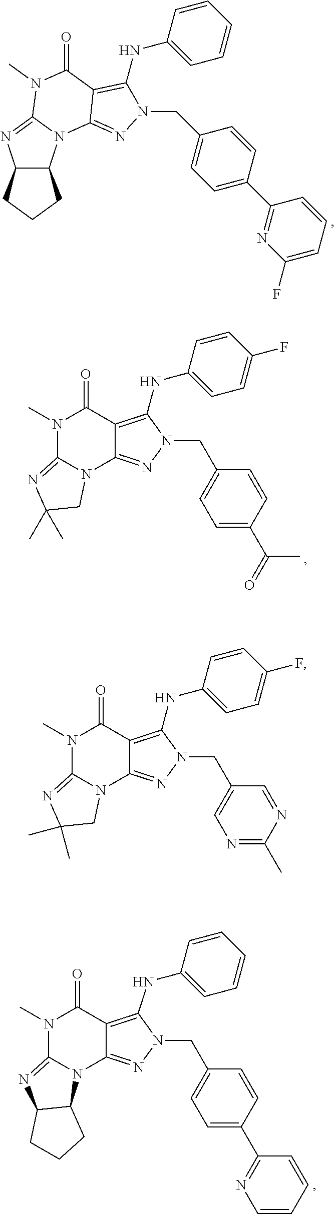

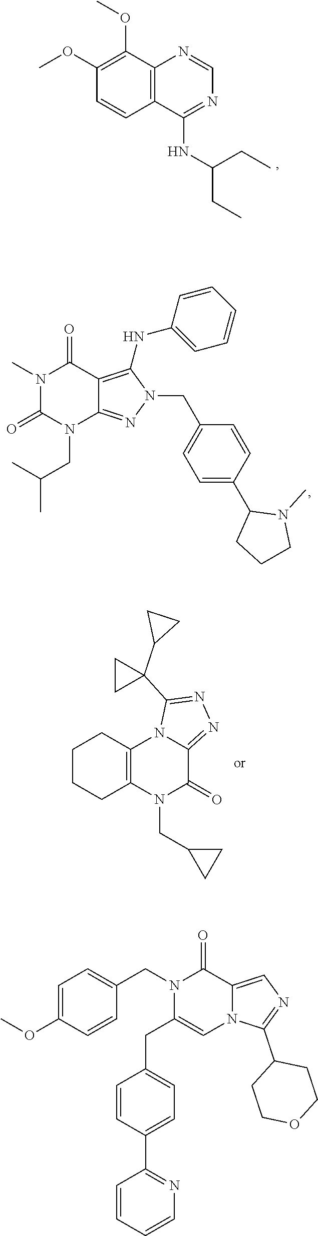

in free or pharmaceutically acceptable salt form.
1.19 Any of the preceding Methods, wherein the PDE1 inhibitor is a compound according to Formulas I, Ia, II, III, IV, V, and/or VI in free or pharmaceutically acceptable salt form.
1.20 Any of the preceding Methods, wherein the PDE1 inhibitor comprises (6aR,9aS)-5,6a, 7,8,9,9a-hexahydro-5-methyl-3-(phenylamino)-2-((4-(6-fluoropyridin-2-yl)phenyl)methyl)-cyclopent [4,5]imidazo[1,2-a]pyrazolo[4,3-e]pyrimidin-4(2H)-one:

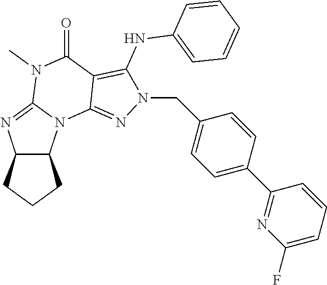 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
1.21 Any of the preceding Methods, wherein the PDE1 inhibitor comprises 7,8-dihydro-2-(4-acetylbenzyl)-3-(4-fluorophenylamino)-5, 7,7-trimethyl-[2H]-imidazo-[1,2-a]pyrazolo[4, 3-e]pyrimidin-4(5H)-one:

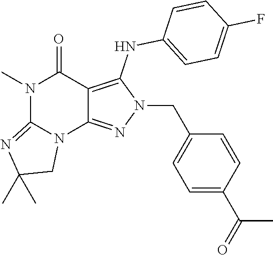 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.
1.22 Any of the preceding Methods, wherein the PDE1 inhibitor comprises 3-((4-fluorophenyl) amino)-5,7,7-trimethyl-2-((2-methylpyrimidin-5-yl)methyl)-7,8-dihydro-2H-imidazo[1, 2-a]pyrazolo[4,3-e]pyrimidin-4(5H)-one:

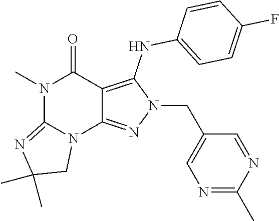 [SMILES: Cc1ncc(Cn2nc3c(c2Nc2ccc(F)cc2)C(=O)N(C)C2=NC(C)(C)CN23)cn1]

in free or pharmaceutically acceptable salt form.
1.23 Any of the preceding Methods, wherein the PDE1 inhibitor comprises (6aR,9aS)-5,6a, 7,8,9,9a-hexahydro-5-methyl-3-(phenylamino)-2-((4-(pyridin-2-yl)phenyl)methyl)-cyclopent[4, 5]imidazo[1,2-a]pyrazolo[4,3-e]pyrimidin-4(2H)-one:

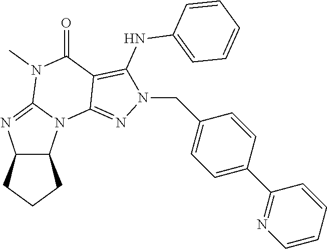 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.

The disclosure further provides a PDE1 inhibitor for use in a method for the treatment or prophylaxis of a renal disorder, e.g., for use in any of Methods 1, et seq.

The disclosure further provides the use of a PDE1 inhibitor in the manufacture of a medicament for the treatment or prophylaxis of a renal disorder, e.g., a medicament for use in any of Methods 1, et seq.

### Methods of Making Compounds of the Disclosure

The PDE1 inhibitors of the Disclosure and their pharmaceutically acceptable salts may be made using the methods as described and exemplified in U.S. Pat. No. 8,273,750, US 2006/0173878, U.S. Pat. No. 8,273,751, US 2010/0273753, U.S. Pat. Nos. 8,697,710, 8,664,207, 8,633,180, 8,536,159, US 2012/0136013, US 2011/0281832, US 2013/0085123, US 2013/0324565, US 2013/0338124, US 2013/0331363, WO 2012/171016, and WO 2013/192556, and by methods similar thereto and by methods known in the chemical art. Such methods include, but not limited to, those described below. If not commercially available, starting materials for these processes may be made by procedures, which are selected from the chemical art using techniques which are similar or analogous to the synthesis of known compounds.

Various PDE1 inhibitors and starting materials therefor may be prepared using methods described in US 2008-0188492 A1, US 2010-0173878 A1, US 2010-0273754 A1, US 2010-0273753 A1, WO 2010/065153, WO 2010/065151, WO 2010/065151, WO 2010/065149, WO 2010/065147, WO 2010/065152, WO 2011/153129, WO 2011/133224, WO 2011/153135, WO 2011/153136, WO 2011/153138. All references cited herein are hereby incorporated by reference in their entirety.

The Compounds of the Disclosure include their enantiomers, diastereomers and racemates, as well as their polymorphs, hydrates, solvates and complexes. Some individual compounds within the scope of this disclosure may contain double bonds. Representations of double bonds in this disclosure are meant to include both the E and the Z isomer of the double bond. In addition, some compounds within the scope of this disclosure may contain one or more asymmetric centers. This disclosure includes the use of any of the optically pure stereoisomers as well as any combination of stereoisomers.

It is also intended that the Compounds of the Disclosure encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the Compounds of the Disclosure may be replaced with deuterium (a stable isotope which is non-radioactive). Examples of known stable isotopes include, but not limited to, deuterium, ¹³ C, ¹⁵N, ¹⁸O. Alternatively, unstable isotopes, which are radioactive isotopes which contain additional neutrons compared to the abundant nuclides of the same species (i.e., element), e.g., ¹²³I, ¹³¹I, ¹²⁵I, ¹¹C, ¹⁸F, may replace the corresponding abundant species of I, C and F. Another example of useful isotope of the compound of the disclosure is the ¹¹C isotope. These radio isotopes are useful for radio-imaging and/or pharmacokinetic studies of the compounds of the disclosure.

Melting points are uncorrected and (dec) indicates decomposition. Temperature are given in degrees Celsius (° C.); unless otherwise stated, operations are carried out at room or ambient temperature, that is, at a temperature in the range of 18-25 ° C. Chromatography means flash chromatography on silica gel; thin layer chromatography (TLC) is carried out on silica gel plates. NMR data is in the delta values of major diagnostic protons, given in parts per million (ppm) relative to tetramethylsilane (TMS) as an internal standard. Conventional abbreviations for signal shape are used. Coupling constants (J) are given in Hz. For mass spectra (MS), the lowest mass major ion is reported for molecules where isotope splitting results in multiple mass spectral peaks Solvent mixture compositions are given as volume percentages or volume ratios. In cases where the NMR spectra are complex, only diagnostic signals are reported.

The words “treatment” and “treating” are to be understood accordingly as embracing treatment or amelioration of symptoms of disease as well as treatment of the cause of the disease.

For methods of treatment, the word “effective amount” is intended to encompass a therapeutically effective amount to treat a specific disease or disorder.

The term “patient” include human or non-human (i.e., animal) patient. In particular embodiment, the disclosure encompasses both human and nonhuman. In another embodiment, the disclosure encompasses nonhuman. In other embodiment, the term encompasses human.

The term “comprising” as used in this disclosure is intended to be open-ended and does not exclude additional, un-recited elements or method steps.

Dosages employed in practicing the present disclosure will of course vary depending, e.g. on the particular disease or condition to be treated, the particular Compounds of the Disclosure used, the mode of administration, and the therapy desired. Compounds of the Disclosure may be administered by any suitable route, including orally, parenterally, transdermally, or by inhalation, but are preferably administered orally. In general, satisfactory results, e.g. for the treatment of diseases as hereinbefore set forth are indicated to be obtained on oral administration at dosages of the order from about 0.01 to 2.0 mg/kg. In larger mammals, for example humans, an indicated daily dosage for oral administration of both the PDE1 inhibitor will accordingly be in the range of from about 0.50 to 300 mg, conveniently administered once, or in divided doses 2 to 4 times, daily or in sustained release form. Unit dosage forms for oral administration thus for example may comprise from about 0.2 to 150 or 300 mg, e.g. from about 0.2 or 2.0 to 10, 25, 50, 75 100, 150, or 200 mg of a Compound of the Disclosure, together with a pharmaceutically acceptable diluent or carrier therefor.

Compounds of the Disclosure may be administered by any satisfactory route, including orally, parenterally (intravenously, intramuscular or subcutaneous) or transdermally, but are preferably administered orally. In certain embodiments, the Compounds of the Disclosure, e.g., in depot formulation, is preferably administered parenterally, e.g., by injection.

The Compounds of the Disclosure and the Pharmaceutical Compositions of the Disclosure of the Disclosure may be used in combination with one or more additional therapeutic agents, particularly at lower dosages than when the individual agents are used as a monotherapy so as to enhance the therapeutic activities of the combined agents without causing the undesirable side effects commonly occur in conventional monotherapy. Therefore, the Compounds of the Disclosure may be simultaneously, separately, sequentially, or contemporaneously administered with other agents useful in treating disease. In another example, side effects may be reduced or minimized by administering a Compound of the Disclosure in combination with one or more additional therapeutic agents in free or salt form, wherein the dosages of (i) the second therapeutic agent(s) or (ii) both Compound of the Disclosure and the second therapeutic agent, are lower than if the agent/compound are administered as a monotherapy. By way of non-limiting example, such additional therapeutic agents may include ACE inhibitors, Angiotensin II receptor antagonists, calcium channel blockers, etc.

The term “simultaneously” when referring to a therapeutic use means administration of two or more active ingredients at or about the same time by the same route of administration.

The term “separately” when referring to a therapeutic use means administration of two or more active ingredients at or about the same time by different route of administration.

Pharmaceutical compositions comprising Compounds of the Disclosure may be prepared using conventional diluents or excipients and techniques known in the galenic art. Thus, oral dosage forms may include tablets, capsules, solutions, suspensions and the like.

### EXAMPLES

Measurement of PDEIB inhibition in vitro using IMAP Phosphodiesterase Assay Kit

Phosphodiesterase I B (PDEIB) is a calcium/calmodulin dependent phosphodiesterase enzyme that converts cyclic guanosine monophosphate (cGMP) to 5′-guanosine monophosphate (5′-GMP). PDEIB can also convert a modified cGMP substrate, such as the fluorescent molecule cGMP-fluorescein, to the corresponding GMP-fluorescein. The generation of GMP-fluorescein from cGMP-fluorescein can be quantitated, using, for example, the IMAP (Molecular Devices, Sunnyvale, CA) immobilized-metal affinity particle reagent.

Briefly, the IMAP reagent binds with high affinity to the free 5′- phosphate that is found in GMP-fluorescein and not in cGMP-fluorescein. The resulting GMP-fluorescein-IMAP complex is large relative to cGMP-fluorescein. Small fluorophores that are bound up in a large, slowly tumbling, complex can be distinguished from unbound fluorophores, because the photons emitted as they fluoresce retain the same polarity as the photons used to excite the fluorescence.

In the phosphodiesterase assay, cGMP-fluorescein, which cannot be bound to IMAP, and therefore retains little fluorescence polarization, is converted to GMP-fluorescein, which, when bound to IMAP, yields a large increase in fluorescence polarization (Amp). Inhibition of phosphodiesterase, therefore, is detected as a decrease in Amp.

### Enzyme Assay

Materials: All chemicals are available from Sigma-Aldrich (St. Louis, MO) except for IMAP reagents (reaction buffer, binding buffer, FL-GMP and IMAP beads), which are available from Molecular Devices (Sunnyvale, CA).

Assay: The following phosphodiesterase enzymes may be used: 3′,5′-cyclic- nucleotide-specific bovine brain phosphodiesterase (Sigma, St. Louis, Mo.) (predominantly PDEIB) and recombinant full length human PDE1A and PDE1B (r- hPDE1A and r-hPDE1B respectively) which may be produced e.g., in HEK or SF9 cells by one skilled in the art. The PDE1 enzyme is reconstituted with 50% glycerol to 2.5 U/ml. One unit of enzyme will hydrolyze 1.0 μmol of 3′,5′-cAMP to 5′-AMP per min at pH 7.5 at 30° C. One part enzyme is added to 1999 parts reaction buffer (30 μM CaCl₂, 10 U/ml of calmodulin (Sigma P2277), 10 mM Tris-HCl pH 7.2, 10 mM MgCl₂, 0.1% BSA, 0.05% NaN₃) to yield a final concentration of 1.25 mU/ml. 99 μl of diluted enzyme solution is added into each well in a flat bottom 96-well polystyrene plate to which 1 μl of test compound dissolved in 100% DMSO is added. The compounds are mixed and pre-incubated with the enzyme for 10 min at room temperature.

The FL-GMP conversion reaction is initiated by combining 4 parts enzyme and inhibitor mix with 1 part substrate solution (0.225 μM) in a 384-well microtiter plate. The reaction is incubated in dark at room temperature for 15 min. The reaction is halted by addition of 60 μL of binding reagent (1:400 dilution of IMAP beads in binding buffer supplemented with 1:1800 dilution of antifoam) to each well of the 384-well plate. The plate is incubated at room temperature for 1 hour to allow IMAP binding to proceed to completion, and then placed in an Envision multimode microplate reader (PerkinElmer, Shelton, CT) to measure the fluorescence polarization (Amp).

A decrease in GMP concentration, measured as decreased Amp, is indicative of inhibition of PDE activity. IC50 values are determined by measuring enzyme activity in the presence of 8 to 16 concentrations of compound ranging from 0.0037 nM to 80,000 nM and then plotting drug concentration versus AmP, which allows IC50 values to be estimated using nonlinear regression software (XLFit; IDBS, Cambridge, Mass.).

The Compounds of the Invention are tested in an assay as described or similarly described herein for PDE1 inhibitory activity. For example, Compound 214, is identified as a specific PDE1 inhibitor of formula:

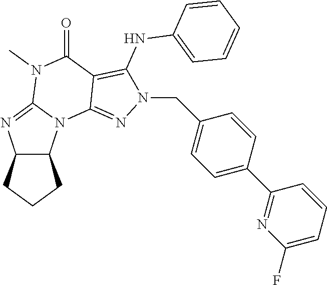 [SMILES: CN1C(=O)c2c(nn(Cc3ccc(-c4cccc(F)n4)cc3)c2Nc2ccccc2)N2C1=N[C@@H]1CCC[C@@H]12]

This compound has efficacy at sub-nanomolar levels vs PDE1 (IC₅₀ of 0.058 nM for bovine brain PDE1 in the assay described above) and high selectivity over other PDE families, as depicted on the following table:

| PDE Target | IC50 (nM) | ratio PDEx/PDE1 |
|---|---|---|
| bovine brain PDE1 | 0.058 | 1 |
| hPDE2A | 3661 | 63121 |
| hPDE3B | 3120 | 53793 |
| hPDE4A | 158 | 2724 |
| r-bovine PDE5A | 632 | 10897 |
| bovine retina PDE6 | 324 | 5586 |
| hPDE7B | 355 | 6121 |
| hPDE8A | 3001 | 51741 |
| hPDE9A | 16569 | 285672 |
| hPDE10A | 1824 | 31448 |
| hPDE11A | 1313 | 22638 |

The compound is also highly selective versus a panel of 63 receptors, enzymes, and ion channels. These data, and data for other PDE1 inhibitors described herein, are described in Li et al., J. Med. Chem. 2016: 59, 1149-1164, the contents of which are incorporated herein by reference.

Alternative combinations and variations of the examples provided will become apparent based on the disclosure. It is not possible to provide specific examples for all of the many possible variations of the embodiments described, but such combinations and variations may be claims that eventually issue.

1. A method for the treatment or prophylaxis of a renal disorder, the method comprising administering a pharmaceutically acceptable amount of a PDE1 inhibitor to a subject in need thereof.

2. The method according to claim 1, wherein the renal disorder is selected from one or more of kidney fibrosis, chronic kidney disease, renal failure, glomerulosclerosis and nephritis.

3. The method according to claim 1, wherein the condition is kidney fibrosis.

4. The method according to claim 1, wherein the condition is chronic kidney disease.

5. The method according to claim 1, wherein the condition is renal failure.

6. The method according to claim 1, wherein the condition is glomerulosclerosis.

7. The method according to claim 1, wherein the condition is nephritis.

8. The method according to claim 1, wherein the renal disorder is consequent to diabetes, an injury to a kidney, high blood pressure, a cancerous growth (e.g., polycystic kidney disease), or a cardiovascular disorder (e.g. angina, stroke, essential hypertension, pulmonary hypertension, secondary hypertension, isolated systolic hypertension, hypertension associated with diabetes, hypertension associated with atherosclerosis, renovascular hypertension, congestive heart failure, myocardial, angina, and stroke, hypertension, an inflammatory disease or disorder, fibrosis, cardiac hypertrophy, vascular remodeling, and an connective tissue disease or disorder, e.g., Marfan Syndrome).

9. The method according to claim 1, wherein the renal disorder is consequent to diabetes.

10. The method according to claim 1, wherein the renal disorder is consequent to an injury to a kidney.

11. The method according to claim 1, wherein the renal disorder is consequent to high blood pressure.

12. The method according to claim 1, wherein the renal disorder is consequent to a cancerous growth (e.g., polycystic kidney disease).

13. The method according to claim 1, wherein the renal disorder is consequent to a cardiovascular disorder (e.g. angina, stroke, essential hypertension, pulmonary hypertension, secondary hypertension, isolated systolic hypertension, hypertension associated with diabetes, hypertension associated with atherosclerosis, renovascular hypertension, congestive heart failure, myocardial, angina, and stroke, hypertension, an inflammatory disease or disorder, fibrosis, cardiac hypertrophy, vascular remodeling, and an connective tissue disease or disorder, e.g., Marfan Syndrome).

14. The method according to claim 1, wherein the PDE1 inhibitor is a compound selected from
(A) Formula I: 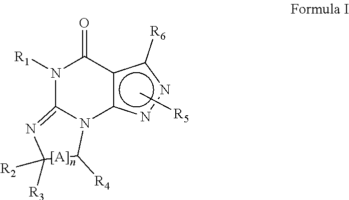
wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero)arylalkoxy, or (optionally hetero)arylalkyl; or
R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl;
or R₅ is attached to one of the nitrogens on the pyrazolo portion of Formula I and is a moiety of Formula A 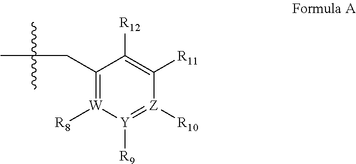
wherein X, Y and Z are, independently, N or C, and R₈, R₉, R¹¹ and R¹² are independently H or halogen (e.g., Cl or F), and R¹⁰ is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl (e.g., benzyl), arylamino (e.g., phenylamino), heterarylamino, N,N-dialkylamino, N,N-diarylamino, or N-aryl-N-(arylalkyl)amino (e.g., N-phenyl-N-(1,1′-biphen-4-ylmethyl)amino); and
(v) n=0 or 1;
(vi) when n=1, A is —C(R₁₃R₁₄)—
wherein R₁₃ and R₁₄, are, independently, H or C₁₋₄ alkyl, aryl, heteroaryl, (optionally hetero)arylalkoxy or (optionally hetero)arylalkyl;
in free, salt or prodrug form, including its enantiomers, diastereoisomers and racemates;
(B) Formula Ia: 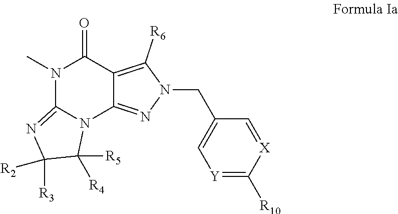
wherein
(i) R₂ and R₅ are independently H or hydroxy and R₃ and R₄ together form a tri- or tetra-methylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref. the carbon carrying R₃ having the R configuration];
(ii) R₆ is (optionally halo- or hydroxy-substituted) phenylamino, (optionally halo- or hydroxy-substituted) benzylamino, C₁₋₄alkyl, or C₁₋₄alkyl sulfide; for example, phenylamino or 4-fluorophenylamino;
(iii) R¹⁰ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and
(iv) X and Y are independently C or N,
in free, pharmaceutically acceptable salt or prodrug form, including its enantiomers, diastereoisomers and racemates;
(C) Formula II: 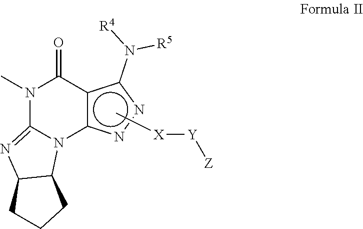
(i) X is C₁₋₆alkylene (e.g., methylene, ethylene or prop-2-yn-l-ylene);
(ii) Y is a single bond, alkynylene (e.g., —C≡C—), arylene (e.g., phenylene) or heteroarylene (e.g., pyridylene);
(iii) Z is H, aryl (e.g., phenyl), heteroaryl (e.g., pyridyl, e.g., pyrid-2-yl), halo (e.g., F, Br, Cl), haloC₁₋₆alkyl (e.g., trifluoromethyl), —C(O )—R¹, —N(R²)(R³), or C₃₋₇cycloalkyl optionally containing at least one atom selected from a group consisting of N or O (e.g., cyclopentyl, cyclohexyl, tetrahydro-2H-pyran-4-yl, or morpholinyl);
(iv) R¹ is C₁₋₆alkyl, haloC₁₋₆alkyl, —OH or —OC₁₋₆alkyl (e.g., —OCH₃);
(v) R² and R³ are independently H or C₁₋₆alkyl;
(vi) R⁴ and R⁵ are independently H, C₁₋₆alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆alkoxy;
(vii) wherein X, Y and Z are independently and optionally substituted with one or more halo (e.g., F, Cl or Br), C₁₋₆alkyl (e.g., methyl), haloC₁₋₆alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl),
in free, salt or prodrug form;
(D) Formula III: 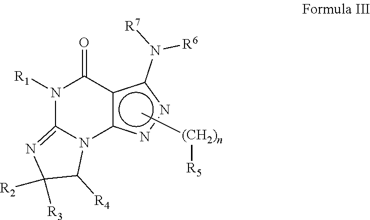
wherein
(i) R₁ is H or C₁₋₄alkyl (e.g., methyl or ethyl);
(ii) R₂ and R₃ are independently H or C₁₋₆alkyl (e.g., methyl or ethyl);
(iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆alkyl (e.g., methyl or ethyl) and halogen (e.g., F or Cl), for example unsubstituted phenyl or phenyl substituted with one or more halogen (e.g., F) or phenyl substituted with one or more C₁₋₆alkyl and one or more halogen or phenyl substituted with one C₁₋₆alkyl and one halogen, for example 4-fluorophenyl or 3,4-difluorophenyl or 4-fluoro-3-methylphenyl; and
(vi) n is 1, 2, 3, or 4,
in free or salt form;
(E) Formula IV 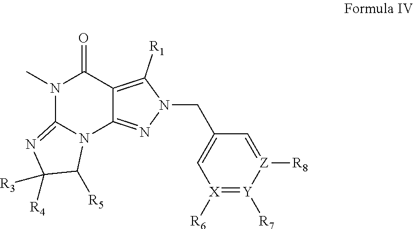
in free or salt form, wherein
(i) R₁ is C₁₋₄alkyl (e.g., methyl or ethyl), or —NH(R₂), wherein R₂ is phenyl optionally substituted with halo (e.g., fluoro), for example, 4-fluorophenyl;
(ii) X, Y and Z are, independently, N or C;
(iii) R₃, R₄ and R₅ are independently H or C₁₋₄alkyl (e.g., methyl); or R₃ is H and R₄ and R₅ together form a tri-methylene bridge (pref. wherein the R₄ and R₅ together have the cis configuration, e.g., where the carbons carrying R₄ and R₅ have the R and S configurations, respectively),
(iv) R₆, R₇ and R₈ are independently:
H,
C₁₋₄alkyl (e.g., methyl),
pyrid-2-yl substituted with hydroxy, or
—S(O)₂—NH₂;
(v) Provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy,
(F) Formula V 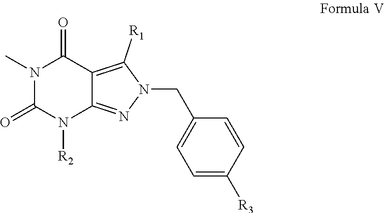
wherein
(i) R₁ is —NH(R₄), wherein R₄ is phenyl optionally substituted with halo (e.g., fluoro), for example, 4-fluorophenyl;
(ii) R₂ is H or C₁₋₆alkyl (e.g., methyl, isobutyl or neopentyl);
(iii) R₃ is —SO₂NH₂ or —COOH;
in free or salt form; and
(G) Formula VI 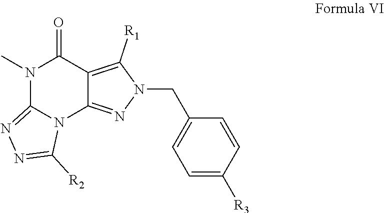
wherein
(i) R₁ is —NH(R₄), wherein R₄ is phenyl optionally substituted with halo (e.g., fluoro), for example, 4-fluorophenyl;
(ii) R₂ is H or C₁₋₆alkyl (e.g., methyl or ethyl);
(iii) R₃ is H, halogen (e.g., bromo), C₁₋₆alkyl (e.g., methyl), aryl optionally substituted with halogen (e.g., 4-fluorophenyl), heteroaryl optionally substituted with halogen (e.g., 6-fluoropyrid-2-yl or pyrid-2-yl), or acyl (e.g., acetyl),
in free or pharmaceutically acceptable salt form.

15. The method according to claim 1, wherein the PDE1 inhibitor is selected from any of the following

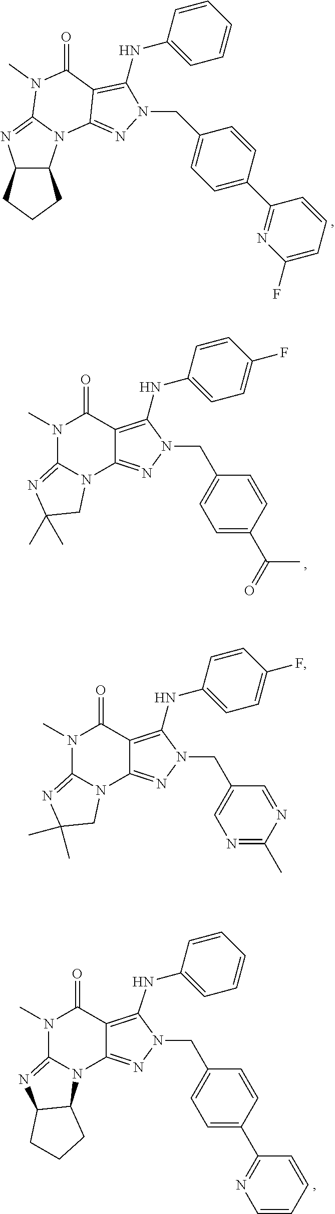

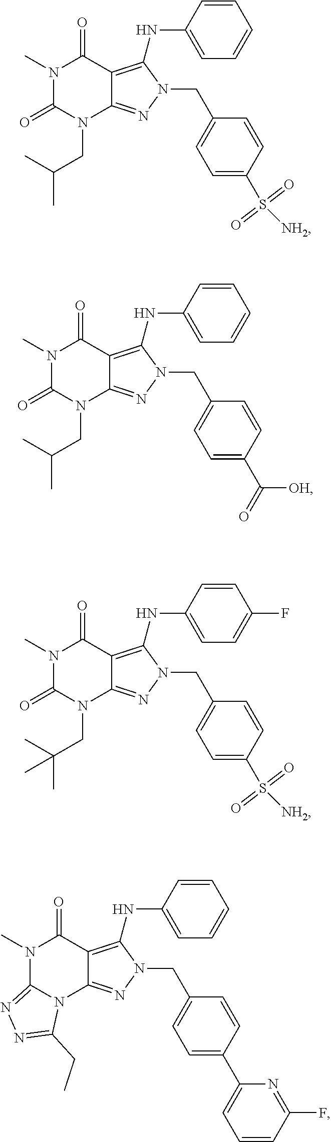 [SMILES: CC(C)Cn1c(=O)n(C)c(=O)c2c(Nc3ccccc3)n(Cc3ccc(S(N)(=O)=O)cc3)nc21 ; CC(C)Cn1c(=O)n(C)c(=O)c2c(Nc3ccccc3)n(Cc3ccc(C(=O)O)cc3)nc21 ; Cn1c(=O)c2c(Nc3ccc(F)cc3)n(Cc3ccc(S(N)(=O)=O)cc3)nc2n(CC(C)(C)C)c1=O ; CCc1nnc2n(C)c(=O)c3c(Nc4ccccc4)n(Cc4ccc(-c5cccc(F)n5)cc4)nc3n12]

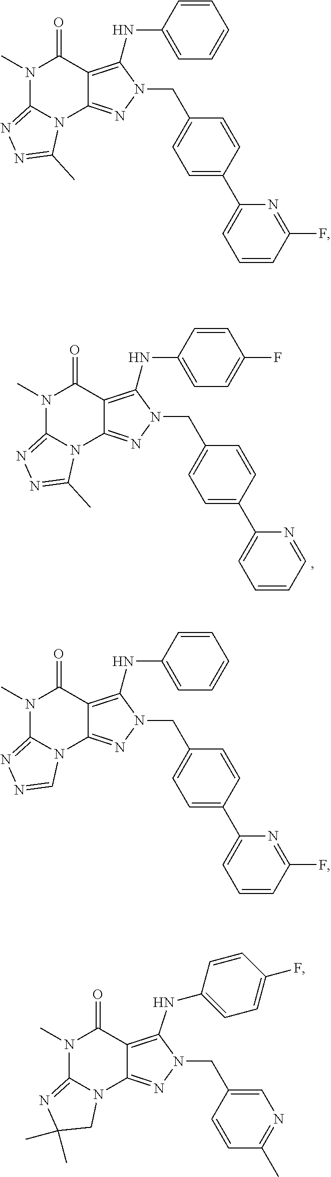 [SMILES: Cc1nnc2n(C)c(=O)c3c(Nc4ccccc4)n(Cc4ccc(-c5cccc(F)n5)cc4)nc3n12 ; Cc1nnc2n(C)c(=O)c3c(Nc4ccc(F)cc4)n(Cc4ccc(-c5ccccn5)cc4)nc3n12 ; Cn1c(=O)c2c(Nc3ccccc3)n(Cc3ccc(-c4cccc(F)n4)cc3)nc2n2cnnc12 ; Cc1ccc(Cn2nc3c(c2Nc2ccc(F)cc2)C(=O)N(C)C2=NC(C)(C)CN23)cn1]

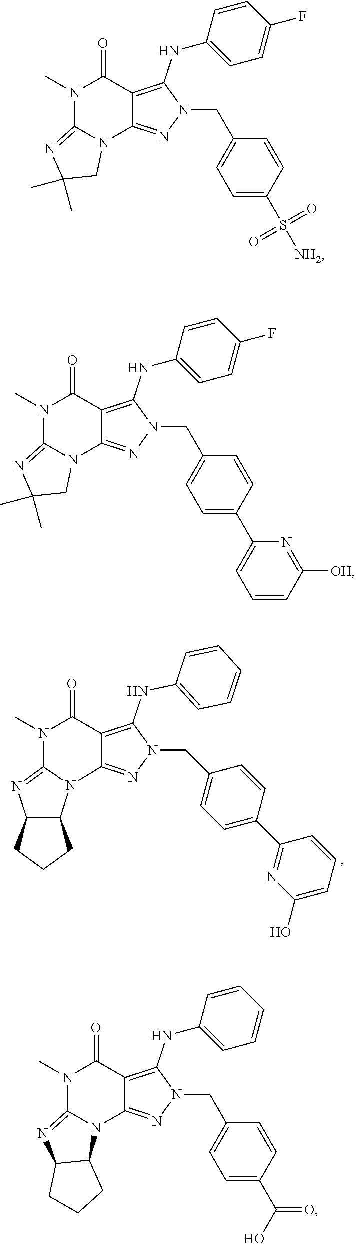 [SMILES: CN1C(=O)c2c(nn(Cc3ccc(S(N)(=O)=O)cc3)c2Nc2ccc(F)cc2)N2CC(C)(C)N=C12 ; CN1C(=O)c2c(nn(Cc3ccc(-c4cccc(O)n4)cc3)c2Nc2ccc(F)cc2)N2CC(C)(C)N=C12 ; CN1C(=O)c2c(nn(Cc3ccc(-c4cccc(O)n4)cc3)c2Nc2ccccc2)N2C1=N[C@@H]1CCC[C@@H]12 ; CN1C(=O)c2c(nn(Cc3ccc(C(=O)O)cc3)c2Nc2ccccc2)N2C1=N[C@@H]1CCC[C@@H]12]

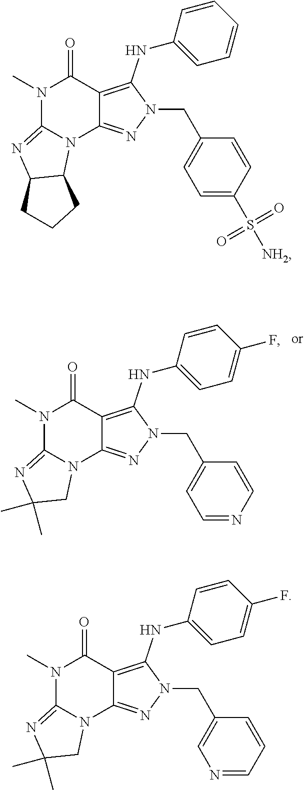 [SMILES: CN1C(=O)c2c(nn(Cc3ccc(S(N)(=O)=O)cc3)c2Nc2ccccc2)N2C1=N[C@@H]1CCC[C@@H]12 ; CN1C(=O)c2c(nn(Cc3ccncc3)c2Nc2ccc(F)cc2)N2CC(C)(C)N=C12 ; "or" ; CN1C(=O)c2c(nn(Cc3cccnc3)c2Nc2ccc(F)cc2)N2CC(C)(C)N=C12]

16. The method according to claim 1, wherein the PDE1 inhibitor is selected from any of the following 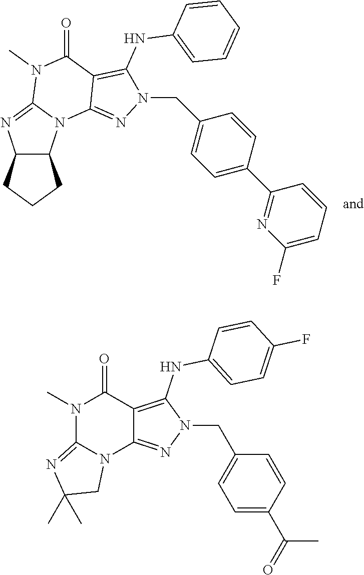 [SMILES: CN1C(=O)c2c(nn(Cc3ccc(-c4cccc(F)n4)cc3)c2Nc2ccccc2)N2C1=N[C@@H]1CCC[C@@H]12 ; CC(=O)c1ccc(Cn2nc3c(c2Nc2ccc(F)cc2)C(=O)N(C)C2=NC(C)(C)CN23)cc1]
in free or pharmaceutically acceptable salt form.

17. A The method according to claim 1, wherein the PDE1 inhibitor is 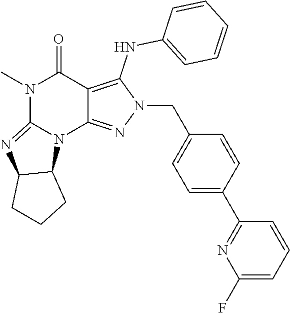 [SMILES: CN1C(=O)c2c(nn(Cc3ccc(-c4cccc(F)n4)cc3)c2Nc2ccccc2)N2C1=N[C@@H]1CCC[C@@H]12]
in free or pharmaceutically acceptable salt form.

18. The method according to claim 1, wherein the PDE1 inhibitor is 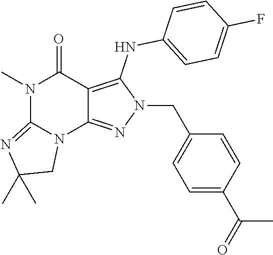 [SMILES: CC(=O)c1ccc(Cn2nc3c(c2Nc2ccc(F)cc2)C(=O)N(C)C2=NC(C)(C)CN23)cc1]
in free or pharmaceutically acceptable salt form.

19. The method of claim 1, wherein the PDE1 inhibitor is the following: 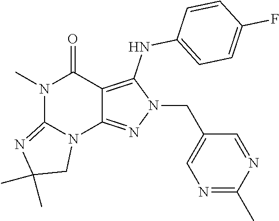 [SMILES: Cc1ncc(Cn2nc3c(c2Nc2ccc(F)cc2)C(=O)N(C)C2=NC(C)(C)CN23)cn1]
in free or pharmaceutically acceptable form.

20. The method of claim 1, wherein the PDE1 inhibitor is the following: 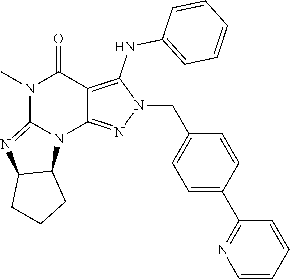 [SMILES: CN1C(=O)c2c(nn(Cc3ccc(-c4ccccn4)cc3)c2Nc2ccccc2)N2C1=N[C@@H]1CCC[C@@H]12]
in free or pharmaceutically acceptable form.

21. (canceled)

22. (canceled)
